(19) United States
(12) Patent Application Publication
(10) Pub. No.: US 2016/0129112 A1
(43) Pub. Date: May 12, 2016

(54) Pharmaceutical Compositions Comprising Pyrophosphate

(71) Applicant: Momenta Pharmaceuticals, Inc., Cambridge, MA (US)

(72) Inventor: Kelly NEELON, Groveland, MA (US)

(21) Appl. No.: 14/891,521

(22) PCT Filed: May 27, 2014
(86) PCT No.: PCT/US2014/039547

§ 371 Date: Nov. 16, 2015

Related U.S. Application Data
(60) Provisional application No. 61/827,924, filed on May 28, 2013.

(51) Int. Cl. A61K 47/02; A61K 31/727; A61K 39/395; C07K 16/18

(52) U.S. Cl. CPC A61K 47/02; C07K 16/18; A61K 31/727; A61K 39/39591

(57) ABSTRACT

Described herein are therapeutic compositions comprising a therapeutic agent and pyrophosphate.

### CROSS REFERENCE TO RELATED APPLICATIONS

This application claims the benefit of U.S. Provisional Application Ser. No. 61/827,924, filed on May 28, 2013, the contents of which are herein incorporated by reference in their entirety.

### FIELD OF THE INVENTION

The invention relates to pharmaceutical formulations comprising pyrophosphate and a therapeutic agent such as a polypeptide or polysaccharide.

### BACKGROUND OF INVENTION

Pharmaceutical compositions that include therapeutic agents such as a polypeptide or a polysaccharide are often formulated into an aqueous solution. For the therapeutic agent to remain biologically active, the compositions should preserve the integrity of the therapeutic agent.

### SUMMARY OF INVENTION

Described herein are pharmaceutical compositions that comprise a therapeutic agent, such as a therapeutic polypeptide and/or a therapeutic polysaccharide, and pyrophosphate. In some embodiments the pharmaceutical composition is an aqueous composition.

In one aspect, the disclosure features an aqueous pharmaceutical composition, the composition comprising: (a) a therapeutic agent, e.g., a therapeutic polypeptide or a therapeutic polysaccharide; and (b) pyrophosphate. In some embodiments, the composition comprises about 5 mM to about 250 mM pyrophosphate, e.g., about 5 mM to about 200 mM, about 5 mM to about 150 mM, about 5 mM to about 100 mM, or about 5 mM to about 50 mM pyrophosphate. In some embodiments, the composition comprises at least about 5 mM pyrophosphate, e.g., at least about 10 mM, about 20 mM, about 30 mM, about 40 mM, about 50 mM, about 60 mM, about 70 mM, about 80 mM, about 90 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 450 mM, or about 500 mM pyrophosphate.

In some embodiments, the therapeutic agent is a therapeutic polypeptide. In some embodiments, the therapeutic polypeptide is an antibody.

In some embodiments, the antibody is selected from the group consisting of: an anti-TNF antibody; an anti-T cell CD3 receptor antibody; an anti-CD25 antibody; an anti-CD20 antibody; an anti-IL-2Rα receptor antibody; an anti-IL-1β antibody; an anti-ErbB2 antibody; an anti-CD33 antibody; an anti-CD52 antibody; an anti-CD11a antibody; an anti-EGFR antibody; an anti-VEGF antibody; an anti-IgE antibody; an anti-α4 integrin antibody; an anti-VEGFRA antibody; an anti-VEGFRB antibody; an anti-RANK ligand antibody; an anti-IL-6R antibody; an anti-CD30 antibody; and an anti-CTLA4 antibody.

In some embodiments, the antibody is a recombinant humanized, chimeric or human antibody. In some embodiments, the antibody is an IgG selected from the group consisting of: an IgG1, an IgG2, an IgG3 and an IgG4.

In some embodiments, the therapeutic agent is a therapeutic antibody preparation (e.g., abciximab, adalimumab, alemtuzumab, basiliximab, bevacizumab, certolizumab, cetuximab, daclizumab, eculizumab, efalizumab, gemtuzumab, ibritumomab, infliximab, muromonab-CD3, natalizumab, omalizumab, palivizumab, panitumumab, ranibizumab, rituximab, tositumomab, or trastuzumab).

In some embodiments, the composition comprises a therapeutic polypeptide at a concentration of from about 1 mg/ml to about 150 mg/ml (e.g., from about 20 mg/ml to about 130 mg/ml; from about 25 to about 100 mg/ml; from about 30 to about 75 mg/ml; or about 40 mg/ml). In some embodiments, the composition comprises a therapeutic polypeptide at a concentration of at least about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 185 mg/ml, about 190 mg/ml, about 195 mg/ml, or about 200 mg/ml.

In some embodiments, the therapeutic agent is a therapeutic polysaccharide. In some embodiments, the therapeutic polysaccharide is a heparin (e.g., an unfractionated heparin (UFH) or a low molecular weight heparin (LMWH)). In some embodiments, the therapeutic polysaccharide is a LMWH selected from the group consisting of: enoxaparin, dalteparin, adomiparin, and necuparinol.

In some embodiments, the composition comprises less than about 1% phosphate, e.g., less than about 0.9%, less than about 0.8%, less than about 0.7%, less than about 0.6%, less than about 0.5%, less than about 0.4%, less than about 0.3%, less than about 0.2%, less than about 0.1%, less than about 0.075%, less than about 0.05%, less than about 0.025%, or less than about 0.01% phosphate. In some embodiments, the composition comprises less than about 5000 ppm, less than about 2500 ppm, or less than about 1000 ppm phosphate.

In some embodiments, the composition has a pH of from about 4 to about 8 (e.g., from about 4.5 to about 6; from about 4.8 to about 5.5; or from about 5.0 to about 5.2).

In some embodiments, the composition further comprises one or more of citrate, acetate, phosphate, succinate, and malate.

In some embodiments, the composition further comprises an excipient. In some embodiments, the excipient is or comprises a surfactant. In some embodiments, the surfactant is or comprises a polysorbate (e.g., polysorbate 80) or a TWEEN (e.g., TWEEN 80).

In some embodiments, the excipient is or comprises a polyol. In some embodiments, the polyol is mannitol or sorbitol.

In some embodiments, the excipient is or comprises a lyoprotectant. In some embodiments, the excipient is or comprises a salt. In some embodiments, the salt is NaCl.

In some embodiments, the excipient is or comprises a preservative. In some embodiments, the composition is substantially free of a preservative.

In some embodiments, the composition is stable, when exposed to a freeze thaw cycle (e.g., at least 2, 3, 4, 5, 6, or more freeze thaw cycles).

In some embodiments, the composition is stable for at least 1 month at a temperature from about 2° C. to about 8° C. (e.g., for at least about 3 months, 6 months, 9 months, 12 months 18 months, 24 months, 30 months, 36 months, or longer).

In some embodiments, the composition is isotonic.

In another aspect, the invention features an aqueous pharmaceutical composition, the composition comprising: (a) a therapeutic agent, e.g., a therapeutic polypeptide or a therapeutic polysaccharide; (b) pyrophosphate; (c) a buffer; (d) a polyol; and (e) a surfactant.

In some embodiments, the composition comprises a therapeutic polypeptide at a concentration of from about 1 mg/ml to about 150 mg/ml (e.g., from about 20 mg/ml to about 130 mg/ml; from about 25 to about 100 mg/ml; from about 30 to about 75 mg/ml; or about 40 mg/ml). In some embodiments, the composition comprises a therapeutic polypeptide at a concentration of at least about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 185 mg/ml, about 190 mg/ml, about 195 mg/ml, or about 200 mg/ml.

In some embodiments, the composition comprises about 5 mM to about 250 mM pyrophosphate, e.g., about 5 mM to about 200 mM, about 5 mM to about 150 mM, about 5 mM to about 100 mM, or about 5 mM to about 50 mM pyrophosphate. In some embodiments, the composition comprises at least about 5 mM pyrophosphate, e.g., at least about 10 mM, about 20 mM, about 30 mM, about 40 mM, about 50 mM, about 60 mM, about 70 mM, about 80 mM, about 90 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 450 mM, or about 500 mM pyrophosphate.

In some embodiments, the buffer provides a pH of from about 4 to about 8 (e.g., from about 4.5 to about 6; from about 4.8 to about 5.5; or from about 5.0 to about 5.2).

In some embodiments, the composition comprises the polyol (e.g., mannitol) at a concentration of about 5 mg/ml to about 20 mg/ml, e.g., about 5 mg/ml to about 10 mg/ml, about 10 mg/ml to about 15 mg/ml, or about 15 mg/ml to about 20 mg/ml polyol. In some embodiments, the composition comprises the polyol (e.g., mannitol) at a concentration of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, or about 20 mg/ml.

In some embodiments, the composition comprises a surfactant (e.g., a polysorbate, e.g., polysorbate 80) at a concentration of about 0.1 mg/ml to about 10 mg/ml, e.g., about 0.1 mg/ml to about 5 mg/ml, about 5 mg/ml to about 10 mg/ml, about 1 mg/ml to about 10 mg/ml. In some embodiments, the composition comprises a surfactant (e.g., a polysorbate, e.g., polysorbate 80) at a concentration of about 0.1 mg/ml, about 0.5 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, or about 10 mg/ml.

In some embodiments, the therapeutic agent is a therapeutic polypeptide. In some embodiments, the therapeutic polypeptide is an antibody.

In some embodiments, the antibody is selected from the group consisting of: an anti-TNF antibody; an anti-T cell CD3 receptor antibody; an anti-CD25 antibody; an anti-CD20 antibody; an anti-IL-2Rα receptor antibody; an anti-IL-1β antibody; an anti-ErbB2 antibody; an anti-CD33 antibody; an anti-CD52 antibody; an anti-CD11a antibody; an anti-EGFR antibody; an anti-VEGF antibody; an anti-IgE antibody; an anti-α4 integrin antibody; an anti-VEGFRA antibody; an anti-VEGFRB antibody; an anti-RANK ligand antibody; an anti-IL-6R antibody; an anti-CD30 antibody; and an anti-CTLA4 antibody.

In some embodiments, the antibody is a recombinant humanized, chimeric or human antibody. In some embodiments, the antibody is an IgG selected from the group consisting of: an IgG1, an IgG2, an IgG3 and an IgG4.

In some embodiments, the therapeutic agent is a therapeutic antibody preparation (e.g., abciximab, adalimumab, alemtuzumab, basiliximab, bevacizumab, certolizumab, cetuximab, daclizumab, eculizumab, efalizumab, gemtuzumab, ibritumomab, infliximab, muromonab-CD3, natalizumab, omalizumab, palivizumab, panitumumab, ranibizumab, rituximab, tositumomab, or trastuzumab).

In some embodiments, the therapeutic agent is a therapeutic polysaccharide. In some embodiments, the therapeutic polysaccharide is a heparin (e.g., an unfractionated heparin (UFH) or a low molecular weight heparin (LMWH)). In some embodiments, the therapeutic polysaccharide is a LMWH selected from the group consisting of: enoxaparin, dalteparin, adomiparin, and necuparinol.

In some embodiments, the composition comprises less than about 1% phosphate, e.g., less than about 0.9%, less than about 0.8%, less than about 0.7%, less than about 0.6%, less than about 0.5%, less than about 0.4%, less than about 0.3%, less than about 0.2%, less than about 0.1%, less than about 0.075%, less than about 0.05%, less than about 0.025%, or less than about 0.01% phosphate. In some embodiments, the composition comprises less than about 5000 ppm, less than about 2500 ppm, or less than about 1000 ppm phosphate.

In some embodiments, the composition comprises about 20 to about 130 mg/ml of a therapeutic protein; pyrophosphate (e.g., 5 mM-250 mM, e.g., 5 mM-100 mM); a buffer providing a solution having a pH of from about 4 to about 8; about 5 to about 20 mg/ml of a polyol (e.g., mannitol); and about 0.1 to about 10 mg/ml a surfactant (e.g., a polysorbate such as polysorbate 80).

In some embodiments, the composition is stable, when exposed to a freeze thaw cycle (e.g., at least 2, 3, 4, 5, 6, or more freeze thaw cycles).

In some embodiments, the composition is stable for at least 1 month at a temperature from about 2° C. to about 8° C. (e.g., for at least about 3 months, 6 months, 9 months, 12 months 18 months, 24 months, 30 months, 36 months, or longer).

In some embodiments, the composition is isotonic.

In another aspect, the invention features a unit dose of a pharmaceutical composition, the composition comprising an aqueous solution comprising a therapeutic agent, e.g., a therapeutic polypeptide or a therapeutic polysaccharide; and pyrophosphate.

In some embodiments, the composition comprises about 5 mM to about 250 mM pyrophosphate, e.g., about 5 mM to about 200 mM, about 5 mM to about 150 mM, about 5 mM to about 100 mM, or about 5 mM to about 50 mM pyrophosphate. In some embodiments, the composition comprises at least about 5 mM pyrophosphate, e.g., at least about 10 mM, about 20 mM, about 30 mM, about 40 mM, about 50 mM, about 60 mM, about 70 mM, about 80 mM, about 90 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 450 mM, or about 500 mM pyrophosphate.

In some embodiments, the therapeutic agent is a therapeutic polypeptide. In some embodiments, the therapeutic polypeptide is an antibody.

In some embodiments, the antibody is selected from the group consisting of: an anti-TNF antibody; an anti-T cell CD3 receptor antibody; an anti-CD25 antibody; an anti-CD20 antibody; an anti-IL-2Rα receptor antibody; an anti-IL-1β antibody; an anti-ErbB2 antibody; an anti-CD33 antibody; an anti-CD52 antibody; an anti-CD11a antibody; an anti-EGFR antibody; an anti-VEGF antibody; an anti-IgE antibody; an anti-α4 integrin antibody; an anti-VEGFRA antibody; an anti-VEGFRB antibody; an anti-RANK ligand antibody; an anti-IL-6R antibody; an anti-CD30 antibody; and an anti-CTLA4 antibody.

In some embodiments, the antibody is a recombinant humanized, chimeric or human antibody. In some embodiments, the antibody is an IgG selected from the group consisting of: an IgG1, an IgG2, an IgG3 and an IgG4.

In some embodiments, the therapeutic agent is a therapeutic antibody preparation (e.g., abciximab, adalimumab, alemtuzumab, basiliximab, bevacizumab, certolizumab, cetuximab, daclizumab, eculizumab, efalizumab, gemtuzumab, ibritumomab, infliximab, muromonab-CD3, natalizumab, omalizumab, palivizumab, panitumumab, ranibizumab, rituximab, tositumomab, or trastuzumab).

In some embodiments, the therapeutic agent is a therapeutic polysaccharide. In some embodiments, the therapeutic polysaccharide is a heparin (e.g., an unfractionated heparin (UFH) or a low molecular weight heparin (LMWH)). In some embodiments, the therapeutic polysaccharide is a LMWH selected from the group consisting of: enoxaparin, dalteparin, adomiparin, and necuparinol.

In some embodiments, the composition is stable, when exposed to a freeze thaw cycle (e.g., at least 2, 3, 4, 5, 6, or more freeze thaw cycles).

In some embodiments, the composition is stable for at least 1 month at a temperature from about 2° C. to about 8° C. (e.g., for at least about 3 months, 6 months, 9 months, 12 months 18 months, 24 months, 30 months, 36 months, or longer).

In some embodiments, the composition is isotonic.

In another aspect, the invention features a method of making a pharmaceutical composition, the method comprising: (a) providing a therapeutic agent (e.g., a lyophilized or aqueous therapeutic agent), e.g., a therapeutic protein or therapeutic polysaccharide; and (b) combining the therapeutic agent with an aqueous solution comprising pyrophosphate, wherein the aqueous solution does not comprise phosphate, to thereby make a pharmaceutical composition.

In some embodiments, the aqueous solution comprises about 5 mM to about 250 mM pyrophosphate, e.g., about 5 mM to about 200 mM, about 5 mM to about 150 mM, about 5 mM to about 100 mM, or about 5 mM to about 50 mM pyrophosphate. In some embodiments, the composition comprises at least about 5 mM pyrophosphate, e.g., at least about 10 mM, about 20 mM, about 30 mM, about 40 mM, about 50 mM, about 60 mM, about 70 mM, about 80 mM, about 90 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 450 mM, or about 500 mM pyrophosphate.

In some embodiments, the therapeutic agent is a therapeutic polypeptide. In some embodiments, the therapeutic polypeptide is an antibody.

In some embodiments, the antibody is selected from the group consisting of: an anti-TNF antibody; an anti-T cell CD3 receptor antibody; an anti-CD25 antibody; an anti-CD20 antibody; an anti-IL-2Rα receptor antibody; an anti-IL-1β antibody; an anti-ErbB2 antibody; an anti-CD33 antibody; an anti-CD52 antibody; an anti-CD11a antibody; an anti-EGFR antibody; an anti-VEGF antibody; an anti-IgE antibody; an anti-α4 integrin antibody; an anti-VEGFRA antibody; an anti-VEGFRB antibody; an anti-RANK ligand antibody; an anti-IL-6R antibody; an anti-CD30 antibody; and an anti-CTLA4 antibody.

In some embodiments, the antibody is a recombinant humanized, chimeric or human antibody. In some embodiments, the antibody is an IgG selected from the group consisting of: an IgG1, an IgG2, an IgG3 and an IgG4.

In some embodiments, the therapeutic agent is a therapeutic antibody preparation (e.g., abciximab, adalimumab, alemtuzumab, basiliximab, bevacizumab, certolizumab, cetuximab, daclizumab, eculizumab, efalizumab, gemtuzumab, ibritumomab, infliximab, muromonab-CD3, natalizumab, omalizumab, palivizumab, panitumumab, ranibizumab, rituximab, tositumomab, or trastuzumab).

In some embodiments, the therapeutic agent is a therapeutic polysaccharide. In some embodiments, the therapeutic polysaccharide is a heparin (e.g., an unfractionated heparin (UFH) or a low molecular weight heparin (LMWH)). In some embodiments, the therapeutic polysaccharide is a LMWH selected from the group consisting of: enoxaparin, dalteparin, adomiparin, and necuparinol.

In some embodiments, the composition is stable, when exposed to a freeze thaw cycle (e.g., at least 2, 3, 4, 5, 6, or more freeze thaw cycles).

In some embodiments, the composition is stable for at least 1 month at a temperature from about 2° C. to about 8° C. (e.g., for at least about 3 months, 6 months, 9 months, 12 months 18 months, 24 months, 30 months, 36 months, or longer).

In some embodiments, the composition is isotonic.

In another aspect, the invention features a method of administering a pharmaceutical composition to a subject, the method comprising parenterally administering a composition described herein, e.g., a composition comprising a therapeutic agent and pyrophosphate, to a subject.

In another aspect, the invention features a method of treating a subject, the method comprising administering to a subject in need thereof a therapeutically effective amount of a composition described herein, e.g., a composition comprising a therapeutic agent and pyrophosphate. In some embodiments, the administering comprises administering parenterally.

In another aspect, the invention features an article of manufacture, e.g., a device, a syringe, a pen, or a vial, comprising an aqueous pharmaceutical composition described herein, e.g., a composition comprising a therapeutic agent and pyrophosphate.

### DETAILED DESCRIPTION OF THE INVENTION

Pharmaceutical compositions, methods of making pharmaceutical compositions, and uses thereof are described herein. In general, the pharmaceutical compositions described herein are aqueous compositions, which are administered to a subject to treat a disorder. Also included herein are dry compositions that include a therapeutic agent and pyrophosphate. The dry composition can be reconstituted into an aqueous solution to provide a pharmaceutical composition described herein.

### Pharmaceutical Compositions

The pharmaceutical compositions described herein include a therapeutic agent, such as a therapeutic polypeptide (e.g., an antibody) and/or a therapeutic polysaccharide, and pyrophosphate. In general, the pharmaceutical compositions described herein are stable.

A “stable” composition, as referred to herein, is one in which the therapeutic agent such as a therapeutic polypeptide, therapeutic polysaccharide, or small molecule retains its chemical integrity (e.g., is substantially free from degradation products) for 30 days after formulation, at standard storage conditions for the composition (e.g., 2° C.-8° C.). Chemical integrity can be assessed by detecting and/or quantifying altered forms of the therapeutic agent. Exemplary altered forms of the therapeutic agent include aggregates and degraded agents. “Substantially free from degradation products” means that the preparation contains less than 10% (e.g., less than 5%, less than 2%, less than 1%, less than 0.5%, less than 0.1%, less than 0.01%) degradation products.

In embodiments where the therapeutic agent is a therapeutic polypeptide, such as an antibody, chemical integrity can be determined by detecting and quantifying the presence or amount of aggregation as measured by size exclusion chromatography. For example, a “stable” composition is one wherein less than about 10% and preferably less than about 5%, 2% or 1% of the therapeutic agent such as a polypeptide (e.g., an antibody) aggregates within 30 days of storage at standard storage conditions (e.g., 2° C.-8° C.).

In embodiments where the therapeutic agent is a therapeutic polysaccharide, such as a LMWH, chemical integrity can be determined by detecting and quantifying the presence or amount of degradation products by NMR, e.g., 1D NMR. For example, a “stable” glycol split LMWH composition is one wherein the preparation contains less than 10%, less than 5%, less than 2%, less than 1%, less than 0.5%, less than 0.1%, less than 0.01% by weight (weight/weight) of formic acid as detected by NMR, e.g., 1D NMR, or levels of formic acid that are undetectable by NMR, e.g., 1D NMR, within 30 days, 60 days, or 90 days of storage at standard storage conditions (e.g., 2° C.-8° C.).

In embodiments where the therapeutic agent is a small molecule, chemical integrity can be determined by detecting and quantifying the presence or amount of degradation products by chromatography, e.g., HPLC. For example, a “stable” composition is one wherein less than about 10% and preferably less than about 5%, 2% or 1% of the small molecule forms a degradation product within 30 days at standard storage conditions (e.g., 2° C.-8° C.).

The pharmaceutical compositions described herein have a shelf life of at least 30 days, e.g., at least two months, at least three months, at least six months, at least nine months, twelve months, or at least eighteen months).

By “isotonic” is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

“Pyrophosphate,” as used herein, is an anion, salt, or ester of pyrophosphoric acid. Pyrophosphate is also sometimes referred to as diphosphate, as pyrophosphate contains two phosphate groups covalently bound together. The difference in number of phosphate groups between phosphate and pyrophosphate results in a different number of available negative charges. A phosphate molecule can have up to three negative charges, whereas a pyrophosphate can have up to four negative charges. Pyrophosphates, in general, are highly water soluble and stable at isotonic and physiological pH. As described herein, pyrophosphates also make good complexing agents, and can be useful to improve the stability of certain compounds, including therapeutic agents such as a therapeutic polypeptide or a therapeutic polysaccharide. In some embodiments, the pharmaceutical compositions described herein are substantially free of phosphate molecules. For example, a pharmaceutical composition described herein can have less than 5000 ppm (0.5%) or less than 2500 ppm (0.25%) or less than 1000 ppm (0.1%) of phosphate, e.g., at the time of formulation.

In some embodiments, the pyrophosphate is a salt. Exemplary salts include sodium, potassium, calcium and magnesium salts. In some embodiments the pyrophosphate is sodium pyrophosphate. Aqueous compositions described herein can have, e.g., 5 mM-250 mM pyrophosphate, 5 mM-200 mM pyrophosphate, 5 mM-150 mM pyrophosphate, 5 mM-100 mM pyrophosphate.

### Therapeutic Agents

The pharmaceutical compositions described herein include a therapeutic agent. When the pharmaceutical composition is an aqueous composition the therapeutic agent generally has a concentration in solution of at least about 1 mg/ml, e.g., about 10 to about 500 mg/ml, about 10 to about 400 mg/ml, about 10 to about 300 mg/ml, about 10 to about 200 mg/ml, about 10 to 150 mg/ml, about 20 to about 120 mg/ml, from about 30 to about 80 mg/ml, e.g., about 50 mg/ml.

Exemplary therapeutic agents include therapeutic polypeptides and therapeutic polysaccharides.

### Therapeutic Polypeptides

In certain embodiments, the therapeutic agent is a polypeptide. The terms “polypeptide”, “protein” and “peptide” are used interchangeably herein. Exemplary polypeptides include, but are not limited to: a protease, a kinase, an oxidoreductase, a transferase, a hydrolase, a lyase, an isomerase, a ligase, a permease, a cytokine, a chemokine, a regulatory protein, a chaperone protein, a signal transduction protein, a cytoskelatal protein, a receptor, a transcription factor, a lipid binding protein, a transport protein, a nuclear transport protein, a carrier protein, a nucleic acid binding protein, a secretory protein, a structural protein, a cell surface protein, a membrane protein, an extracellular protein, a protein that mediates electron transport, a protein that mediates ion transport, a protein that mediates transcription, a protein that mediates translation, a protein that mediates in cell death, a protein that mediates apoptosis, a protein that has metabolic activity, a protein that has antioxidant activity, a protein that has integrase activity, a protein that has helicase activity, a protein that functions in cell motility, or a protein that has aromatase activity.

Antibodies

In some embodiments, the polypeptide is an antibody. The term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab')₂, Fd fragments, Fv fragments, scFv, and domain antibodies (dAb) fragments (de Wildt et al., Eur J Immunol. 1996; 26(3):629-39.)) as well as complete antibodies. An antibody can have the structural features of IgA, IgG, IgE, IgD, IgM (as well as subtypes thereof). Antibodies may be from any source, but primate (human and non-human primate) and primatized are preferred. In some embodiments, the antibody is an IgG, e.g., an IgG1, and IgG2, an IgG3 or an IgG4.

As used herein, an “immunoglobulin variable domain sequence” refers to an amino acid sequence which can form the structure of an immunoglobulin variable domain such that one or more complementarity determining regions (CDRs) are positioned in a conformation suitable for an antigen binding site.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab')₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated CDR that retains functionality.

The antibody can be, e.g., a CDR-grafted antibody, a humanized antibody or a human antibody. A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

The antibody can be, e.g., an antibody that binds to one or more of the following antigens: renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-beta; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

Preferred antigens for the antibodies included in the pharmaceutical compositions described herein include: CD proteins (e.g., CD3, CD4, CD8, CD11a, CD19, CD20, CD25, CD33, CD34 and CD52); members of the ErbB receptor family (e.g., EGF receptor, HER2, HER3 or HER4 receptor); cell adhesion molecules (e.g., LFA-1, Mac1, p150,95, VLA-4, ICAM-1, VCAM and αv/β3 integrin including (e.g., anti-CD11a, anti-CD18or anti-CD11b antibodies)); growth factors (e.g., VEGF); a cytokine (e.g., TNF-α, TNF-β); IgE, CTLA-4, and an interleukin (e.g., IL-1, IL-8).

The antibody can be selected from the group consisting of: an anti-TNF antibody; an anti-T cell CD3 receptor antibody; an anti-CD25 antibody; an anti-CD20 antibody; an anti-IL-2Rα receptor antibody; an anti-IL-1β antibody; an anti-ErbB2 antibody; an anti-CD33 antibody; an anti-CD52 antibody; an anti-CD11a antibody; an anti-EGFR antibody; an anti-VEGF antibody; an anti-IgE antibody; an anti-α4 integrin antibody; an anti-VEGFRA antibody; an anti-VEGFRB antibody; an anti-RANK ligand antibody; an anti-IL-6R antibody; an anti-CD30 antibody; and an anti-CTLA4 antibody.

Exemplary, nonlimiting antibodies include abciximab (ReoPro®, Roche), adalimumab (Humira®, Bristol-Myers Squibb), alemtuzumab (Campath®, Genzyme/Bayer), basiliximab (Simulect®, Novartis), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denosumab (Prolia®, Amgen; Xgeva®, Amgen), eculizumab (Soliris®, Alexion Pharmaceuticals), efalizumab (Raptiva®, Genentech), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rituximab (MabThera®, Roche), tocilizumab (Actemra®, Genentech; RoActemra, Hoffman-La Roche), tositumomab (Bexxar®, GlaxoSmithKline), and trastuzumab (Herceptin®, Roche).

Polypeptide Conjugates

In some embodiments, the polypeptide is conjugated or fused to one or more heterologous moieties. Heterologous moieties include, but are not limited to, peptides, polypeptides, proteins, fusion proteins, nucleic acid molecules, small molecules, mimetic agents, synthetic drugs, inorganic molecules, and organic molecules. In some instances, a polypeptide conjugate is a fusion protein that comprises a peptide, polypeptide, protein scaffold, scFv, dsFv, diabody, Tandab, or an antibody mimetic fused to an immunoglobulin Fc region. A fusion protein can include a linker region connecting an Fc region to a heterologous moiety (see, e.g., Hallewell et al. (1989), J. Biol. Chem. 264, 5260-5268; Alfthan et al. (1995), Protein Eng. 8, 725-731; Robinson & Sauer (1996)).

Exemplary, nonlimiting polypeptide conjugates include abatacept (Orencia®, Bristol-Myers Squibb), aflibercept (Eylea®, Regeneron Pharmaceuticals), alefacept (Amevive®, Astellas Pharma), belatacept (Nulojix®, Bristol-Myers Squibb), denileukin diftitox (Ontak®, Eisai), etanercept (Enbrel®, Amgen-Pfizer), and rilonacept (Arcalyst®, Regeneron Pharmaceuticals).

In some instances, a polypeptide conjugate includes an Fc region conjugated to a heterologous polypeptide of at least 10, at least 20, at least 30, at least 40, at least 50, at least 60, at least 70, at least 80, at least 90 or at least 100 amino acids.

In some instances, a polypeptide conjugate includes an Fc region conjugated to one or more marker sequences, such as a peptide to facilitate purification. A particular marker amino acid sequence is a hexa-histidine peptide, such as the tag provided in a pQE vector (QIAGEN, Inc., 9259 Eton Avenue, Chatsworth, Calif., 91311). Other peptide tags useful for purification include, but are not limited to, the hemagglutinin “HA” tag, which corresponds to an epitope derived from the influenza hemagglutinin protein (Wilson et al., 1984, Cell 37:767) and the “Flag” tag.

In other instances, a polypeptide conjugate includes an Fc region conjugated to a diagnostic or detectable agent. Such fusion proteins can be useful for monitoring or prognosing development or progression of disease or disorder as part of a clinical testing procedure, such as determining efficacy of a particular therapy. Such diagnosis and detection can be accomplished by coupling a polypeptide to detectable substances including, but not limited to, various enzymes, such as but not limited to horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as but not limited to iodine (¹³¹I, ¹²⁵I, ¹²³I), carbon (¹⁴C), sulfur (³⁵S), tritium (³H), indium (¹¹⁵In, ¹¹³In, ¹¹²In, ¹¹¹In), technetium (^{99m}Tc), thallium gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵³Gd, ₁₅₉Gd, ¹⁴⁹Pm, ¹⁴⁰La, ¹⁶⁹Yb, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ⁵¹Cr, ⁵⁴Mn, ⁷⁵Se, ¹¹³Sn, and ¹¹⁷Sn; positron emitting metals using various positron emission tomographies, non-radioactive paramagnetic metal ions, and molecules that are radiolabelled or conjugated to specific radioisotopes.

Techniques for conjugating therapeutic moieties to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56. (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987)).

Therapeutic Polysaccharides

In certain embodiments, the pharmaceutical compositions described herein include a therapeutic polysaccharide. A “polysaccharide” as used herein is a polymer composed of monosaccharides linked to one another. In many polysaccharides, the basic building block of the polysaccharide is actually a disaccharide unit, which can be repeating or non-repeating. One or more polysaccharides described herein can be included in a preparation of polysaccharides, e.g., a preparation of a mixed population of polysaccharides, e.g., a heparin preparation, e.g., synthetic heparin preparation, an unfractionated heparin preparation or LMWH preparation. As used herein, a “mixed population of polysaccharides” is a polydisperse mixture of polysaccharides. The term “polydisperse” or “polydispersity” refers to the weight average molecular weight of a preparation (Mw) divided by the number average molecular weight (Mn).

Preferred polysaccharides included in the pharmaceutical compositions described herein are polysaccharides of heparin or heparin sulfate or preparations of polysaccharides derived from heparin or heparin sulfate, e.g., low molecular weight heparins (LMWH). Examples of LMWH polysaccharide preparations that can be included in the disclosed pharmaceutical compositions include, but are not limited to, enoxaparin, dalteparin, certoparin, ardeparin, nadroparin, parnaparin, reviparin, tinzaparin, fondaparinux, adomiparin, and necuparinol.

In one embodiment, the LMWH is a LMWH preparation having the following characteristics: (a) a weight average chain molecular weight between 3,500 and 8,000 Da; (b) anti-Xa activity of less than 20 IU/mg and anti-IIa activity of 1 IU/mg or less (including undetectable anti-IIa activity); (c) greater than 5% and less than 25% glycol split uronic acid residues; and (d) a molecular weight distribution such that 10-40% of the oligosaccharides of the preparation have a molecular weight<3000 Da; 45-65% of the oligosaccharides have a molecular weight between 3000-8000 Da, and 15-30% of the oligosaccharides have a molecular weight>8000 Da. In one embodiment, the LMWH preparation is the LMWH described in WO 2007/140231. In another embodiment, the LMWH preparation is the LMWH described in WO 2011/130572.

### Excipients

The pharmaceutical compositions described herein are generally aqueous solutions. The pharmaceutical compositions described herein can include excipients, such as one or more of a buffer, a bulking agent, surfactant, tonicity agent (e.g., a polyol) preservative, or lyoprotectant. In some embodiments, a dry composition described herein is reconstituted with one or more diluents to result in a pharmaceutical composition described herein such as an aqueous composition.

A “buffer” as used herein is an agent that maintains a stable pH in a solution within a specific pH range. Buffering ranges are determined by pKa. An aqueous formulation can be prepared including the therapeutic agent in a pH-buffered solution. The aqueous solutions generally can have a pH from about a pH of about 4 to about 8 (e.g., from about 4.5 to about 7.5, from about 5.0 to about 7.0, or from about 5.0 to about 5.5, e.g., about 5.2). Examples of buffers include acetate (e.g., sodium acetate), succinate (such as sodium succinate), gluconate, histidine, citrate, malate, phosphate and other organic acid buffers. In some embodiments, the buffer includes a combination of components, such as acetate-phosphate. In a further embodiment, the composition includes sodium hydroxide (e.g., to adjust the pH of the composition). In some embodiments, the buffer comprises citrate.

A “diluent” as used herein is an agent which is pharmaceutically acceptable (safe and non-toxic for administration to a human) and is useful for the preparation of a reconstituted composition. Exemplary diluents include sterile water, bacteriostatic water for injection (BWFI), a pH buffered solution (e.g., phosphate-buffered saline), sterile saline solution, Ringer's solution or dextrose solution.

A “reconstituted” composition is one which has been prepared by dissolving a dry composition (e.g., a composition comprising a dried therapeutic agent such as a lyophilized polypeptide) in a diluent such that the therapeutic agent is dispersed in the reconstituted composition. The reconstituted composition can be suitable for administration (e.g., parenteral administration) to a patient to be treated with the polypeptide of interest and, in certain embodiments, may be one which is suitable for subcutaneous administration.

A “bulking agent,” as used herein, is a compound which adds mass to a lyophilized mixture (such as a dry composition described herein) and can contribute to the physical structure of the lyophilized material such as a lyophilized cake (e.g., facilitates the production of an essentially uniform lyophilized cake which maintains an open pore structure). Exemplary bulking agents include mannitol, glycine, polyethylene glycol and xorbitol. In some embodiments, the bulking agent is mannitol. In one embodiment, the pharmaceutical composition contains between about 1 and about 20 mg/ml of mannitol as determined in a reconstituted form such as a pharmaceutical composition described herein, for example, between about 5 and about 15 mg/ml (e.g., about 10 or about 12 mg/ml).

In some embodiments, a pharmaceutical composition described herein can include a surfactant. In some embodiments, the surfactant can reduce aggregation of the reconstituted therapeutic agent such as a polypeptide and/or reduce the formation of particulates in the reconstituted composition. The surfactant can be added to a pre-lyophilized composition, a lyophilized composition and/or a reconstituted composition (but preferably the pre-lyophilized composition) as desired. Exemplary surfactants include detergents include nonionic detergents such as polysorbates (e.g., polysorbates 20, 80 etc) or poloxamers (e.g., poloxamer 188). In certain embodiments, the amount of surfactant added is such that it reduces aggregation of the formulated therapeutic agent and/or minimizes the formation of particulates in the formulation and/or reduces adsorption. In certain embodiments, the composition includes a surfactant which is a polysorbate. In another embodiment, the composition contains polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In one embodiment, the composition contains between about 0.1 and about 10 mg/ml of polysorbate 80, more preferably between about 0.5 and about 5 mg/ml (e.g., about 1 mg/ml).

In some embodiments, a composition described herein includes a tonicity agent. A “tonicity agent” as used herein is a compound which renders the composition isotonic. An exemplary tonicity agent is a polyol. A “polyol” is a substance with multiple hydroxyl groups, and includes sugars (reducing and nonreducing sugars), sugar alcohols and sugar acids. Preferred polyols herein have a molecular weight which is less than about 600 kD (e.g., in the range from about 120 to about 400 kD). A “reducing sugar” is one which contains a hemiacetal group that can reduce metal ions or react covalently with lysine and other amino groups in proteins, and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof. When it is desired that the composition is freeze-thaw stable, the polyol is preferably one which does not crystallize at freezing temperatures (e.g., −20° C.) such that it destabilizes the therapeutic agent in the composition. The polyol may also act as a tonicity agent. In some embodiments, the polyol is mannitol. In some embodiments, the mannitol concentration is about 5 to 20 mg/ml. In some embodiments, the concentration of mannitol is about 7.5 to 15 mg/ml. In some embodiments, the concentration of mannitol is about 10-14 mg/ml. In some embodiments, the concentration of mannitol is about 12 mg/ml. In some embodiments, the polyol is sorbitol. A polyol, which can act as a tonicifier and may stabilize the therapeutic agent (e.g., a polypeptide such as an antibody), can be included in a composition described herein. A polyol can be added to the composition in an amount that may vary with respect to the desired isotonicity of the composition. In some embodiments, the aqueous composition is isotonic.

In some embodiments, a pharmaceutical composition described herein can include a preservative. A “preservative” as used herein is a compound which can be added to the diluent to essentially reduce bacterial action in the reconstituted composition, thus facilitating the production of a multi-use reconstituted composition. Examples of preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, allyl parabens such as methyl or propyl paraben, catechol, resorcinol, cyclohexanol, 3-pentanol, and m-cresol. In some embodiments, the preservative is benzyl alcohol.

Also described herein are methods of preparing a pharmaceutical composition described herein. In some embodiments, the method comprises reconstituting a mixture (e.g., a dry mixture such as a lyophilized mixture) of a therapeutic agent such as a polypeptide or polysaccharide in a diluent and pyrophosphate such that the concentration of the therapeutic agent in the reconstituted composition is at least 20 mg/mL (e.g., at least 30 mg/mL, at least 40 mg/mL, or at least 50 mg/mL). In some embodiments, the concentration of the therapeutic agent in the reconstituted composition is about 2-40 times greater than the concentration of the therapeutic agent in the mixture before lyophilization of the therapeutic agent.

In some embodiments, a pharmaceutical composition described herein is prepared using a method comprising the steps of: (a) lyophilizing a mixture of a therapeutic agent such as a polypeptide or polysaccharide; and (b) reconstituting the lyophilized mixture of step (a) in a diluent and pyrophosphate such that the reconstituted composition is isotonic and stable and has a therapeutic agent concentration of at least 20 mg/mL (e.g., at least 30 mg/mL, at least 40 mg/mL, or at least 50 mg/mL). In some embodiments, the concentration of the therapeutic agent in the reconstituted composition is about 2-40 times greater than the concentration of the therapeutic agent in the mixture before lyophilization.

At the desired stage, typically when it is time to administer the therapeutic agent to the patient, a lyophilized composition can be reconstituted with a diluent such that the concentration of the therapeutic agent in the reconstituted composition is at least 20 mg/mL (e.g., at least 30 mg/mL, at least 40 mg/mL, or at least 50 mg/mL), for example from about 50 mg/mL to about 400 mg/mL, from about 80 mg/mL to about 300 mg/mL, or from about 90 mg/mL to about 150 mg/mL. Such high concentrations in the reconstituted composition can be particularly useful where subcutaneous delivery of the reconstituted composition is intended. However, for other routes of administration, such as intravenous administration, lower concentrations of the therapeutic in the reconstituted composition can be prepared (for example from about 5-50 mg/mL, or from about 10-40 mg/mL therapeutic agent in the reconstituted composition).

Reconstitution can take place at a temperature of about 25° C. to ensure complete hydration, although other temperatures can also be used. The time required for reconstitution can depend, e.g., on the type of diluent, amount of excipient(s) and therapeutic agent (e.g., therapeutic polypeptide or therapeutic polysaccharide). Exemplary diluents include sterile water, bacteriostatic water for injection (BWFI), a pH buffered solution (e.g., phosphate-buffered saline), sterile saline solution, Ringer's solution or dextrose solution. The diluent optionally contains a preservative. Exemplary preservatives have been described above. The amount of preservative employed can be determined by assessing different preservative concentrations for compatibility with the therapeutic agent and preservative efficacy testing. In some embodiments, the reconstituted composition has less than 6000 particles per vial which are >10 μm size. Other methods of formulating pharmaceutical compositions are known in the art and are described, for example, in “Remington: The Science and Practice of Pharmacy” (formerly “Remingtons Pharmaceutical Sciences”), Lippincott, Williams & Wilkins, Philadelphia, Pa. (2012).

### Methods of Treatment

Also described herein is a method of treating a subject, the method comprising administering to the subject a pharmaceutical composition described herein. Also described herein is a method of treating a subject comprising administering a therapeutically effective amount of a reconstituted composition disclosed herein to a subject, wherein the subject has a disorder requiring treatment with the pharmaceutical composition. For example, the pharmaceutical composition may be administered subcutaneously or intravenously.

The terms “treating”, “treatment”, and the like, mean administering the composition to a subject or a cell or tissue of a subject in order to obtain a desired pharmacological, physiological or clinical effect. Treatment with a polysaccharide preparation described herein may lessen, reduce, mitigate, ameliorate, delay, or prevent an existing unwanted condition or the onset or a symptom thereof. A “therapeutically effective amount” refers to an amount effective, at dosages and for periods of time necessary, to achieve the desired pharmacological, physiological or clinical effect in the subject. A “subject” refers to a human or non-human animal.

The pharmaceutical composition can be administered to a subject in need of treatment with the therapeutic agent, such as a human, in accord with known methods, such as intravenous administration as a bolus or by continuous infusion over a period of time, by intramuscular, intraperitoneal, intracerobrospinal, subcutaneous, intra-articular, intrasynovial, and intrathecal routes. In an embodiment, the pharmaceutical composition is administered to the subject by subcutaneous (i.e., beneath the skin) administration. For such purposes, the composition may be injected using a syringe. However, other devices for administration of the composition are available such as injection devices (e.g., the Inject-ease™ and Genject™ devices); injector pens (such as the GenPen™); needleless devices (e.g., MediJector™ and BioJector™); and subcutaneous patch delivery systems. In some embodiments, the pharmaceutical composition is administered to the subject by intravenous administration, e.g., as a bolus or by continuous infusion over a period of time.

The appropriate dosage (“therapeutically effective amount”) of the therapeutic agent will depend, for example, on the condition to be treated, the severity and course of the condition, whether the therapeutic agent is administered for preventive or therapeutic purposes, previous therapy, the patient's clinical history and response to the protein, the type of therapeutic agent used, and the discretion of the attending physician. The therapeutic agent is suitably administered to the patient at one time or over a series of treatments and may be administered to the patient at any time from diagnosis onwards. The therapeutic agent may be administered as the sole treatment or in conjunction with other drugs or therapies useful in treating the condition in question.

Also described herein is an article of manufacture (e.g., a glass or plastic vial, bottle, syringe, or other delivery device) comprising a composition described herein. In one embodiment, the article of manufacture comprises, e.g.: (a) a container which holds a lyophilized mixture of a therapeutic agent; and (b) instructions for reconstituting the lyophilized mixture into a formulation described herein.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1

### Aqueous Compositions

An aqueous composition comprising a model recombinant IgG was prepared as follows:

| Component | Amount |
|---|---|
| Model IgG | 50 mg |
| sodium chloride | 5.67 mg |
| sodium citrate dihydrate | 0.31 mg |
| citric acid monohydrate | 1.31 mg |
| mannitol | 12 mg |
| polysorbate 80 | 1.0 mg |
| sodium pyrophosphate-10-hydrate | 3.75 mg (7.05 mM) |

H₂O and NaOH to 1 mL at pH 5.2

### Example 2

### Evaluation of Stability

Samples of about 250 μL of the composition of Example 1 were provided in a borosilicate type I glass containers and subjected to 3 month stability studies.

After 3 months, the composition remained opalescent, colorless and free of particulate matter. In addition, there were no changes from time 0 in pH, osmolality, protein concentration or A320/A400 values.

Degradation by aggregation and clipping were analyzed by size exclusion chromatography (SEC). No clipping or aggregation was detected at 2° C.-8° C. after three months. At 30° C., the aggregation rate was 0.19%/month (R²=0.98) and at 40° C., the aggregation rate was 0.44%/month (R²=0.98). At 30° C., the clipping rate was 0.43%/month (R²=0.98) and at 40° C., the clipping rate was 0.90%/month (R²=0.98).

Degradation by acidic species generation was analyzed by WCX-10. No significant change was seen at 2° C.-8° C. after three months. At 30° C., the acidic species generation rate was 5.3%/month (R²=0.97) and at 40° C., it was 12.2%/month (R²=0.98).

This example demonstrates that the test composition of Example 1 can be used to stably formulate a polypeptide composition.

### Example 3

### Aqueous Composition

Another example of a stable, aqueous composition of the disclosure, comprising a model recombinant IgG, was prepared as follows:

| Excipient | Amount (mg/ml) | Amount (mM) |
|---|---|---|
| Sodium Citrate dihydrate | 1.305 | 6.8 |
| Citric Acid, monohydrate | 0.305 | 1.0 |
| Sodium Pyrophosphate, 10-hydrate | 6.3 | 14.1 |
| Sodium Chloride | 6.0 | 105 |
| Mannitol | 12.0 | 66 |
| Polysorbate 80 | 1.0 | 0.1% |
| IgG | 50 | N/A |

### Example 4

### Aqueous Formulation of a LMWH

M402, a low molecular weight heparin described in Zhou et al. (PLoS ONE 6(6):e21106. doi:10.1371/journal.pone.0021106 (2011)), was formulated at 150 mg/mL in either 10 mM sodium pyrophosphate or 30 mM sodium pyrophosphate, in water for injection (WFI) and samples were subjected to stability studies. Impurities or degradation products of each sample were determined by SAX analysis at time 0, 15 days, one month and two months. The percent impurities in each sample is shown below.

% Impurities (breakdown products)

| Formulation | Time 0 | 15 days | 1 month | 2 months |
|---|---|---|---|---|
| 10 mM Sodium | 0.5 | 0.6 | 1.0 | 0.9 |
| Pyrophosphate | | | | |
| 30 mM Sodium | 0.1 | 0.5 | 1.2 | 1.2 |
| Pyrophosphate | | | | |

This example demonstrates that pyrophosphate can be used to stably formulate an oligosaccharide composition.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. An aqueous pharmaceutical composition, the composition comprising
a. a therapeutic agent, e.g., a therapeutic polypeptide or a therapeutic polysaccharide; and
b. pyrophosphate, e.g., 5 mM-250 mM pyrophosphate.

2. The composition of claim 1, wherein the therapeutic agent is a therapeutic polypeptide.

3. The composition of claim 2, wherein the therapeutic polypeptide is an antibody.

4. The composition of claim 3, wherein the antibody is selected from the group consisting of: an anti-TNF antibody; an anti-T cell CD3 receptor antibody; an anti-CD25 antibody; an anti-CD20 antibody; an anti-IL-2Rα receptor antibody; an anti-IL-1β antibody; an anti-ErbB2 antibody; an anti-CD33 antibody; an anti-CD52 antibody; an anti-CD11a antibody; an anti-EGFR antibody; an anti-VEGF antibody; an anti-IgE antibody; an anti-α4 integrin antibody; an anti-VEGFRA antibody; an anti-VEGFRB antibody; an anti-RANK ligand antibody; an anti-IL-6R antibody; an anti-CD30 antibody; and an anti-CTLA4 antibody.

5. The composition of claim 3, wherein the antibody is a recombinant humanized, chimeric or human antibody.

6. The composition of claim 3, wherein the antibody is an IgG selected from the group consisting of: an IgG1, an IgG2, an IgG3 and an IgG4.

7. The composition of claim 2, comprising the therapeutic polypeptide at a concentration of from 1 mg/ml to about 150 mg/ml (e.g., from about 20 mg/ml to about 130 mg/ml; from about 25 to about 100 mg/ml; from about 30 to about 75 mg/ml; or about 40 mg/ml).

8. The composition of claim 1, wherein the therapeutic agent is a therapeutic polysaccharide.

9. The composition of claim 8, wherein the therapeutic polysaccharide is a heparin (e.g., an unfractionated heparin (UFH) or a low molecular weight heparin (LMWH)).

10. The composition of claim 8, wherein the therapeutic polysaccharide is a LMWH selected from the group consisting of: enoxaparin, dalteparin, adomiparin, and necuparinol.

11. The composition of claim 1, wherein the composition comprises less than about 0.5% phosphate.

12. The composition of claim 1, wherein the pH of the composition is from about 4 to about 8 (e.g., from about 4.5 to about 6; from about 4.8 to about 5.5; or from about 5.0 to about 5.2).

13. The composition of claim 1, further comprising one or more of citrate, acetate, phosphate, succinate, malate, or mixtures thereof.

14. The composition of claim 1, further comprising an excipient.

15. The composition of claim 14, wherein the excipient comprises a surfactant.

16. The composition of claim 15, wherein the surfactant comprises a polysorbate (e.g., polysorbate 80) or a TWEEN (e.g., TWEEN 80).

17. The composition of claim 14, wherein the excipient comprises a polyol.

18. The composition of claim 17, wherein the polyol is mannitol or sorbitol.

19. The composition of claim 14, wherein the excipient comprises a lyoprotectant.

20. The composition of claim 14, wherein the excipient comprises a salt.

21. The composition of claim 20, wherein the salt is NaCl.

22. The composition of claim 14, wherein the excipient comprises a preservative.

23. The composition of claim 1, wherein the composition is substantially free of a preservative.

24. The composition of claim 1, wherein the composition is stable, when exposed to a freeze thaw cycle (e.g., at least three freeze thaw cycles).

25. The composition of claim 1, wherein the composition is stable for at least 1 month at a temperature from about 2 to about 8° C. (e.g., for at least about 12 months or 18 months).

26. The composition of claim 1, wherein the composition is isotonic.

27. An aqueous pharmaceutical composition, the composition comprising:
a. a therapeutic agent, e.g., a therapeutic protein or a therapeutic polysaccharide;
b. pyrophosphate;
c. a buffer;
d. a polyol; and
e. a surfactant.

28. The aqueous pharmaceutical composition of claim 27, wherein the composition comprises:
a. about 20 to about 130 mg/ml of a therapeutic protein;
b. pyrophosphate (e.g., 5 mM-250 mM, e.g., 5 mM-100 mM);
c. a buffer providing a solution having a pH of from about 4 to about 8;
d. about 5 to about 20 mg/ml of a polyol (e.g., mannitol); and
e. about 0.1 to about 10 mg/ml a surfactant (e.g., a polysorbate such as polysorbate 80).

29. A unit dose of a pharmaceutical composition, the composition comprising an aqueous solution comprising;
a. a therapeutic agent, e.g., a therapeutic protein or therapeutic polysaccharide; and
b. pyrophosphate, e.g., 5 mM-250 mM.

30. A method of making a pharmaceutical composition, the method comprising:
a. providing a therapeutic agent (e.g., lyophilized or aqueous), e.g., a therapeutic protein or therapeutic polysaccharide; and
b. combining the therapeutic agent with an aqueous solution comprising pyrophosphate, wherein the aqueous solution does not comprise phosphate, to thereby make a pharmaceutical composition.

31. A method of administering a pharmaceutical composition to a subject, the method comprising parenterally administering the composition of any one of claims 1-28 to a subject.

32. A method of treating a subject, the method comprising administering to a subject in need thereof a therapeutically effective amount of the composition of any one of claims 1-28.

33. The method of claim 32, wherein the administering comprises administering parenterally.

34. An article of manufacture, e.g., a syringe, a pen, a vial, comprising the aqueous pharmaceutical composition of any one of claims 1-28.

35. The composition of any one of claims 1-28, wherein the therapeutic agent is a therapeutic antibody preparation (e.g., abciximab, adalimumab, alemtuzumab, basiliximab, bevacizumab, certolizumab, cetuximab, daclizumab, eculizumab, efalizumab, gemtuzumab, ibritumomab, infliximab, muromonab-CD3, natalizumab, omalizumab, palivizumab, panitumumab, ranibizumab, rituximab, tositumomab, or trastuzumab).
